(19) United States
(12) Patent Application Publication
(10) Pub. No.: US 2008/0102133 A1
(43) Pub. Date: May 1, 2008

(54) Oral Pharmaceutical Preparation for Proton Pump Antagonists

(76) Inventors: Antje Brueck-Scheffler, Konstanz (DE); Isabel Anstett-Klein, Leinfelden-Echterdingen (DE); Simone Hiltl, Konstanz (DE); Hartmut Ney, Konstanz (DE)

Correspondence Address: NATH & ASSOCIATES PLLC, 112 South West Street, Alexandria, VA, 22314, US

(21) Appl. No.: 11/663,994

(22) PCT Filed: Sep. 30, 2005
(86) PCT No.: PCT/EP05/54953

§ 371 Date: Apr. 30, 2007

(30) Foreign Application Priority Data
Oct. 5, 2004 (EP) 04104883.6

(51) Int. Cl. A61K 9/14; A61K 31/44

(52) U.S. Cl. 424/490; 514/336

(57) ABSTRACT

The invention relates to novel dosage forms for proton pump antagonists.

### TECHNICAL FIELD

The present invention relates to oral pharmaceutical preparations in the form of an orodispersible dosage form for proton pump antagonists.

### STATE OF THE ART

Irreversible proton pump inhibitors (H⁺/K⁺-ATPase inhibitors, PPIs), especially pyridin-2-ylmethyl-sulphinyl-1H-midazoles as disclosed for example in EP-A-0 005 129, EP-A-0 166 287, EP-A-0 174 726 and EP-A-0 268 956, have, by reason of their H⁺/K⁺-ATPase-inhibiting effect, importance in the therapy of diseases derived from increased gastric acid secretion. Irreversible proton pump inhibitors are substances which bind covalently, and thus irreversibly, to the enzyme responsible for acid secretion in the stomach, the H⁺/K⁺-ATPase [description of the mechanism of action for example in Wurst et al., The Yale Journal of Biology and Medicine 69, (1996), 233-243]. Examples of commercially available active ingredients from this group are 5-methoxy-2-[(4-methoxy-3,5-dimethyl-2-pyridinyl)methylsulphinyl ]-1H-benzimidazole (INN: omeprazole), 5-difluoromethoxy-2-[(3,4-dimetho-xy-2-pyridinyl)methylsulphinyl ]-1H-benzimidazole (INN: pantoprazole), 2-[3-methyl-4-(2,2,2-trifluoro-ethoxy)-2-pyridinyl)methylsulphinyl ]-1H-benzimidazole (INN: lansoprazole) and 2-{[4-(3-methoxy-propoxy)-3-methylpyridin-2-yl ]methylsulphinyl}-1H-benzimidazole (INN: rabeprazole).

Besides the so-called irreversible proton pump inhibitors which, as mentioned at the outset, essentially have a common basic chemical structure (they are pyridinylmethylsulphinylbenzimidazoles), there are so-called reversible H⁺/K⁺-ATPase inhibitors which have different basic chemical structures and which—as indicated by the name—reversibly bind to the enzyme responsible for gastric acid secretion and are therefore also called proton pump antagonists or APAs (=acid pump antagonists) [description of the mechanism of action for example in Wurst et al, The Yale Journal of Biology and Medicine 69 (1996), 233-243]. Reversible proton pump inhibitors are disclosed for example in the documents DE-A 3917232, EP-A-0399267, EP-A-0387821, JP-A-3031280, JP-A-2270873, EP-A-0308917, EP-A-0268989, EP-A-0228006, EP-A-0204285, EP-A-0165545, EP-A-0125756, EP-A-0120589, EP-A-0509974, DE-A 3622036, EP-A-0537532, EP-A-0535529, JP-A-3284686, JP-A-3284622, U.S. Pat. No. 4,833,149, EP-A-0261912, WO-A-9114677, WO-A-9315055, WO-A-9315071, WO-A-9315056, WO-A-9312090, WO-A-9212969, WO-A-9118887, EP-A-0393926, EP-A-0307078, U.S. Pat. No. 5,041,442, EP-A-0266890, WO-A-9414795, EP-A-0264883, EP-A-0033094, EP-A-0259174, EP-A-0330485, WO-A-8900570, EP-A-0368158, WO-A-9117164, WO-A-9206979, WO-A-9312090, WO-A-9308190, WO-A-9418199, DE-A 3011490, U.S. Pat. No. 4,464,372, EP-A-0068378 and WO-A-9424130.

EP 0841904 B1 describes an oral pharmaceutical composition for reversible proton pump inhibitors in combination with antimicrobial active ingredients for the treatment of a disease caused by helicobacter. At least part of the reversible proton pump inhibitor is in extended release form.

WO-A-95/27714 is related to substituted tricyclic imidazo[1,2-a]pyridines which reversibly inhibit exogenously or endogenously stimulated gastric acid secretion. On page 38 an example for a tablet formulation is disclosed.

WO-A-0245693 discloses new preparations for an active ingredient, wherein the active ingredient is present essentially uniformly dispersed in an excipient matrix composed of one or more excipients selected from the group of fatty alcohol, triglyceride, partial glyceride and fatty acid ester. It is mentioned that the matrix is inter alia suitable for active ingredients from the class of substances known as reversible propton pump inhibitors or APAs (acid pump antagonists). Rapidly disintegrating tablets based on these preparations are mentioned.

### DESCRIPTION OF THE INVENTION

Providing proton pump antagonists in the form of an orodispersible dosage form may improve the therapeutic effect of the proton pump antagonist in the prevention or treatment of gastrointestional disorders. In particular a faster onset of action and a faster relieve of symptoms (elimination of pain) may be observed in the therapy of gastrointestinal disorders. The present invention therefore relates to an oral dosage form for proton pump antagonists (APA) comprising an effective amount of a proton pump antagonist together with excipients, which dosage form is an orodispersible dosage form.

In particular the oral dosage form is a dosage form wherein the active ingredient (proton pump antagonist) is stabilized in the dosage form by basic excipients.

Another aspect of the invention is therefore a stable oral dosage form for reversible proton pump inhibitors comprising an effective amount of a proton pump antagonist (APA) together with excipients, where the proton pump antagonist is stabilized in the dosage form by one or more basic excipients and wherein the dosage form is an orodispersible dosage form.

Irreversible proton pump inhibitors (H⁺/K⁺-ATPase inhibitors, PPIs) are according to the invention substances which are able to bind covalently, and thus irreversibly, to the enzyme responsible for acid secretion in the stomach, H⁺/K⁺-ATPase [description of the possible mechanism of action for example in Wurst et al., The Yale Journal of Biology and Medicine 69, 3, 1996, 233-243]. By this are meant in particular pyridin-2-yl-methylsulphinyl-1H-benzimidazoles as disclosed for example in EP-A-0 005 129, EP-A-0 166 287, EP-A-0 174 726 and EP-A-0 268 956. Examples which may be mentioned are 5-methoxy-2-[(4-methoxy-3,5-dimethyl-2-pyridinyl)methylsulphinyl]-1H-benzimidazole (INN: omeprazole), 5-difluoromethoxy-2-[(3,4-dimethoxy-2-pyridinyl)methylsulphinyl]-1H-benzimidazole (INN: pantoprazole), 2-[3-methyl-4-(2,2,2-trifluoroethoxy)-2-pyridinyl)methylsulphinyl ]-1H-benzimidazole (INN: lansoprazole) and 2-{[4-(3-methoxypropoxy)-3-methylpyridin-2-yl]methylsulphinyl}-1H-benzimidazole (INN: rabeprazole).

Proton pump antagonists, also called according to the invention reversible proton pump inhibitors or APA (acid pump antagonists), are for the purposes of the present invention those active ingredients able to bind reversibly to the enzyme responsible for gastric acid secretion H⁺/K⁺-ATPase [description of the possible mechanism of action of the APAs for example in Wurst et al, The Yale Journal of Biology and Medicine 69, 3, 1996, 233-243]. The term proton pump antagonists includes according to the invention not only the active ingredient as such but also the pharmacologically acceptable salts and solvates (especially hydrates) etc. Examples of proton pump antagonists are mentioned in the following documents:

EP 33094, EP 204285, EP 228006, EP 233760, EP 259174, EP 266326, EP 266890, EP 270091, EP 307078, EP 308917, EP 330485, U.S. Pat. No. 4,728,658, U.S. Pat. No. 5362743, WO 9212969, WO 9414795, WO 9418199, WO 9429274, WO 9510518, WO 9527714, WO 9603405, WO 9604251, WO 9605177, WO 9703074, WO 9703076, WO 9747603, WO 9837080, WO 9842707, WO 9843968, WO 9854188, WO 9909029, WO 9928322, WO 9950237, WO 9951584, WO 9955705, WO 9955706, WO 0001696, WO 0010999, WO 0011000, WO 0017200, WO 0026217, WO 0029403, WO 0063211, WO 0077003, WO 0158901, WO 0172754, WO 0172755, WO 0172756, WO 0172757, WO 02034749, WO 02060440, WO 02060441 and WO 02060442.

Examples of proton pump antagonists which may be mentioned by means of their INNs or their code designation are the compounds: AG-2000 (EP 233760), AU-461 (WO 9909029), BY112 (WO 9842707), soraprazan (BY359) (WO 0017200), CP-113411 (U.S. Pat. No. 5,362,743), DBM-819 (WO 0001696), KR-60436 (WO 9909029), pumaprazole (WO 9418199), SKF-96067 (EP 259174), SKF-96356 (EP 307078), SKF-97574 (EP 330485), T-330 (EP 270091), T-776 (EP 270091), WY-27198 (U.S. Pat. No. 4,728,658), YH-1885 (WO 9605177), YJA-20379-8 (WO 9703074), YM-19020 (EP 266890) and 2,3-dimethyl-8-(2-ethyl-6-methylbenzylamino)imidazo[1,2-a]pyridine-6-carboxamide (WO 02060440).

Particularly worthy of mention in this connection are the compounds AU-461, soraprazan (BYK61359), DBM-819, KR-60436, T-330, YH-1885, YJA-20379-8 and 2,3-dimethyl-8-(2-ethyl-6-methylbenzylamino)imidazo[1,2-a ]pyridine-6-carboxamide.

A group of APAs which is of particular interest according to the invention is described and claimed in the patent applications WO 9842707, WO 9854188, WO 0017200, WO 0026217, WO 0063211, WO 0172754, WO 0172755, WO 0172756, WO 0172757, WO 02034749, WO03014120, WO03016310, WO03014123, WO03068774 and WO03091253.

Examples of APAs which may be mentioned in connection with the invention are the following compounds:

(7S,8R,9R)-2,3-dimethyl-7,8-dihydroxy-9-phenyl-7,8,9,10-tetrahydroimidazo[1,2-h][1,7]naphthyridine,

(7R,8R,9R)-3-hydroxymethyl-7,8-dihydroxy-2-methyl-9-phenyl-7,8,9,10-tetrahydroimidazo[1,2-h][1,7] naphthyridine,

(7S,8R,9R)-7,8-isopropylidenedioxy-2,3-dimethyl-9-phenyl-7,8,9,10-tetrahydroimidazo[1,2-h][1,7] naphthyridine,

7,8-dihydroxy-9-phenyl-2,3-dimethyl-7H-8,9-dihydropyrano[2,3-c]imidazo[1,2-a]pyridine,

(7R, 8R, 9R)-2,3-dimethyl-8-hydroxy-7-methoxy-9-phenyl-7,8,9,10-tetrahydroimidazo[1,2-h][1,7] naphthyridine,

(7S, 8S, 9S)-2,3-dimethyl-8-hydroxy-7-methoxy-9-phenyl-7,8,9,10-tetrahydroimidazo[1,2-h][1,7] naphthyridine,

(7S, 8R, 9R)-2,3-dimethyl-8-hydroxy-7-methoxy-9-phenyl-7,8,9,10-tetrahydroimidazo[1,2-h][1,7] naphthyridine,

(7R, 8S, 9S)-2,3-dimethyl-8-hydroxy-7-methoxy-9-phenyl-7,8,9,10-tetrahydroimidazo[1,2-h][1,7] naphthyridine,

(7R, 8R, 9R)-2,3-dimethyl-7-ethoxy-8-hydroxy-9-phenyl-7,8,9,10-tetrahydroimidazo[1,2-h][1,7] naphthyridine,

(7S, 8R, 9R)-2,3-dimethyl-7-ethoxy-8-hydroxy-9-phenyl-7,8,9,10-tetrahydroimidazo[1,2-h][1,7] naphthyridine,

(7R, 8R, 9R)-2,3-dimethyl-8-hydroxy-7-(2-methoxyethoxy)-9-phenyl-7,8,9,10-tetrahydroimidazo[1,2-h][1,7]naphthyridine,

(7S, 8S, 9S)-2,3-dimethyl-8-hydroxy-7-(2-methoxyethoxy)-9-phenyl-7,8,9,10-tetrahydroimidazo[1,2-h][1,7]naphthyridine,

(7S, 8R, 9R)-2,3-dimethyl-8-hydroxy-7-(2-methoxyethoxy)-9-phenyl-7,8,9,10-tetrahydroimidazo[1,2-h][1,7]naphthyridine,

(7R, 8S, 9S)-2,3-dimethyl-8-hydroxy-7-(2-methoxyethoxy)-9-phenyl-7,8,9,10-tetrahydroimidazo[1,2-h][1,7]naphthyridine,

(7S, 8R, 9R)-2,3-dimethyl-8-hydroxy-9-phenyl-7-(2-propoxy)-7,8,9,10-tetrahydroimidazo[1,2-h][1,7]-naphthyridine,

(7R,8R,9R)-2,3-dimethyl-7,8-dimethoxy-9-phenyl-7,8,9,10-tetrahydroimidazo[1,2-h][1,7]naphthyridine,

(7R,8R,9R)-2,3-dimethyl-8-hydroxy-7-(2-methylthioethyloxy)-9-phenyl-7,8,9,10-tetrahydroimidazo[1,2-h][1,7]naphthyridine,

(7S,8R,9R)-2,3-dimethyl-8-hydroxy-7-(2-methylthioethyloxy)-9-phenyl-7,8,9,10-tetrahydroimidazo[1,2-h][1,7]naphthyridine,

(7R,8R,9R)-2,3-dimethyl-8-hydroxy-7-(2-methylsulphinylethoxy)-9-phenyl-7,8,9,10-tetrahydroimidazo[1,2-h][1,7]naphthyridine,

(7S,8R,9R)-2,3-dimethyl-8-hydroxy-7-(2-methylsulphinylethoxy)-9-phenyl-7,8,9,10-tetrahydroimidazo[1,2-h][1,7]naphthyridine,

(7R,8R,9R)-2,3-dimethyl-8-hydroxy-7-(ethylthio)-9-phenyl-7,8,9,10-tetrahydroimidazo[1,2-h][1,7] naphthyridine,

(7S,8R,9R)-2,3-dimethyl-8-hydroxy-7-(ethylthio)-9-phenyl-7,8,9,10-tetrahydroimidazo[1,2-h][1,7] naphthyridine,

(7R,8R,9R)-2,3-dimethyl-8-hydroxy-7-(2,2,2-trifluoroethoxy)-9-phenyl-7,8,9,10-tetrahydroimidazo[1,2-h][1,7]naphthyridine,

(7S, 8R, 9R)-2,3-dimethyl-8-hydroxy-7-(2,2,2-trifluoroethoxy)-9-phenyl-7,8,9,10-tetrahydroimidazo[1,2-h][1,7]naphthyridine,

(7S,8R,9R)-8-acetoxy-7-(2-methoxyethoxy)-2,3-dimethyl-9-phenyl-7,8,9,10-tetrahydroimidazo[1,2-h]-[1,7]naphthyridine,

(7R,8R,9R)-8-acetoxy-7-(2-methoxyethoxy)-2,3-dimethyl-9-phenyl-7,8,9,10-tetrahydroimidazo[1,2-h]-[1,7]naphthyridine,

(7R,8R,9R)-8-acetoxy-7-methoxy-2,3-dimethyl-9-phenyl-7,8,9,10-tetrahydroimidazo[1,2-h][1,7] naphthyridine,

(7R,8R,9R)-8-acetoxy-7-ethoxy-2,3-dimethyl-9-phenyl-7,8,9,10-tetrahydroimidazo[1,2-h][1,7] naphthyridine,

(7R,8R,9R)-7-(2-methoxyethoxy)-2,3-dimethyl-9-phenyl-8-propionyloxy-7,8,9,10-tetrahydroimi-dazo[1,2-h][1,7]naphthyridine,

(7R,8R,9R)-8-benzoyloxy-7-(2-methoxyethoxy)-2,3-dimethyl-9-phenyl-7,8,9,10-tetrahydroimidazo[1,2-h][1,7]naphthyridine,

(7S,8R,9R)-8-benzoyloxy-7-(2-methoxyethoxy)-2,3-dimethyl-9-phenyl-7,8,9,10-tetrahydroimidazo[1,2-h][1,7]naphthyridine,

(7R,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,

(7S,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,

(7R,8R,9R)-8-benzoyloxy-7-methoxy-2,3-dimethyl-9-phenyl-7,8,9,10-tetrahydroimidazo[1,2-h][1,7] naphthyridine,

(7S,8R,9R)-8-benzoyloxy-7-methoxy-2,3-dimethyl-9-phenyl-7,8,9,10-tetrahydroimidazo[1,2-h][1,7] naphthyridine,

(7R,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,

(7S,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,

(7S,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,

(7R,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,

(7S,8R,9R)-7-methoxy-2,3-dimethyl-8-(3-nitrobenzoyloxy)-9-phenyl-7,8,9,10-tetrahydroimidazo[1,2-h]-[1,7]naphthyridine,

(7R,8R,9R)-7-methoxy-2,3-dimethyl-8-(3-nitrobenzoyloxy)-9-phenyl-7,8,9,10-tetrahydroimidazo[1,2-h]-[1,7]naphthyridine,

(7S,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,

(7R,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,

(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,

(7S,8R,9R)-7-(2-Methoxyethoxy)-2,3-dimethyl-8-(N,N-dimethylaminomethylcarbonyloxy)-9-phenyl-7,8,9,10-tetrahydroimidazo[1,2-h][1,7]naphthyridine,

(7S,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,

(7R,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,

(7R,8R,9R)-8-ethylaminocarbonyloxy-7-(2-methoxyethoxy)-2,3-dimethyl-9-phenyl-7,8,9,10-tetrahydroimidazo[1,2-h][1,7]naphthyridine,

(7R,8R,9R)-8-benzoyloxy-2,3-dimethyl-7-(2-methoxyethoxy)-9-phenyl-7H-8,9-dihydropyrano[2,3-c]imidazo[1,2-a]pyridine,

(7S,8R,9R)-8-benzoyloxy-2,3-dimethyl-7-(2-methoxyethoxy)-9-phenyl-7H-8,9-dihydropyrano[2,3-c]imidazo[1,2-a]pyridine,

(7R,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,

(7S,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,

(7S,8R,9R)-2,3-dimethyl-7-methoxy-8-methoxyacetyloxy-9-phenyl-7,8,9,10-tetrahydroimidazo[1,2-h][1,7]naphthyridine,

(7R,8R,9R)-8-(N,N-diethylaminocarbonyloxy)-2,3-dimethyl-7-methoxy-9-phenyl-7,8,9,10-tetrahydro-imidazo[1,2-h][1,7]naphthyridine,

(7S,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,

(7R,8R,9R)-7-methoxy-8-methoxycarbonyloxy-2,3-dimethyl-9-phenyl-7,8,9,10-tetrahydroimidazo[1,2-h][1,7]naphthyridine,

(7S,8R,9R)-7-methoxy-8-methoxycarbonyloxy-2,3-dimethyl-9-phenyl-7,8,9,10-tetrahydroimidazo[1,2-h][1,7]naphthyridine,

(7R,8R,9R)-2,3-dimethyl-8-formyloxy-7-methoxy-9-phenyl-7,8,9,10-tetrahydroimidazo[1,2-h][1,7]naphthyridine,

(7S,8R,9R)-2,3-dimethyl-8-formyloxy-7-methoxy-9-phenyl-7,8,9,10-tetrahydroimidazo[1,2-h][1,7]naphthyridine,

(7R,8R,9R)-8-benzoyloxy-2,3-dimethyl-7-methoxy-9-phenyl-7,8,9,10-tetrahydroimidazo[1,2-h][1,7]naphthyridine,

(7R,8S,9R)-2,3,8-trimethyl-7,8-dihydroxy-9-phenyl-7,8,9,10-tetrahydroimidazo[1,2-h][1,7]naphthyridine,

(7S,8S,9R)-2,3-dimethyl-8-benzyl-7,8-dihydroxy-9-phenyl-7,8,9,10-tetrahydroimidazo[1,2-h][1,7]naphthyridine,

(7R,8S,9R)-2,3,8-trimethyl-7,8-0,0-isopropylidene-9-phenyl-7,8,9,10-tetrahydroimidazo[1,2-h][1,7]naphthyridine,

(7S,8S,9R)-2,3,8-trimethyl-7-(2-methoxyethoxy)-8-hydroxy-9-phenyl-7,8,9,10-tetrahydroimidazo[1,2-h][1,7]naphthyridine,

(7S,8S,9R)-2,3,8-trimethyl-7-methoxy-8-hydroxy-9-phenyl-7,8,9,10-tetrahydroimidazo[1,2-h][1,7]naphthyridine,

(7R,8R,9R)-2,3,7-trimethyl-7,8-dihydroxy-9-phenyl-7,8,9,10-tetrahydroimidazo[1,2-h][1,7]naphthyridine,

(7R,8R,9R)-2,3,7-trimethyl-7,8-[1,3]dioxolo-9-phenyl-7,8,9,10-tetrahydroimidazo[1,2-h][1,7] naphthyridine,

(8S,9R)-2,3-dimethyl-8-hydroxy-7-methylidene-9-phenyl-7,8,9,10-tetrahydroimidazo[1,2-h][1,7] naphthyridine,

(7S,8R,9R)-2,3,7-trimethyl-7,8-dihydroxy-9-phenyl-7H-8,9-dihydropyrano[2,3-c]imidazo[1,2-a]pyridine,

(7R,8R,9R)-2,3,7-trimethyl-7,8-dihydroxy-9-phenyl-7H-8,9-dihydropyrano[2,3-c]imidazo[1,2-a]pyridine,

(7S,8R,9R)-2,3-dimethyl-7,8-dihydroxy-7,9-diphenyl-7H-8,9-dihydropyrano[2,3-c]imidazo[1,2-a]pyridine,

(7S,8R,9R)-2,3-dimethyl-7-(2′,2′-dimethylvinyl)-7,8-dihydroxy-9-phenyl-7H-8,9-dihydropyrano[2,3-c] imidazo[1,2-a]pyridine,

(7R,8R,9R)-2,3-dimethyl-7,8-O-isopropylidene-9-phenyl-7-vinyl-7H-8,9-dihydropyrano[2,3-c] imidazo[1,2-a]pyridine,

(7R,8R,9R)-2,3-dimethyl-8-hydroxy-7-(2-methoxyethoxy)-9-phenyl-7H-8,9-dihydropyrano[2,3-c] imidazo[1,2-a]pyridine,

(7S,8R,9R)-2,3-dimethyl-8-hydroxy-7-(2-methoxyethoxy)-9-phenyl-7H-8,9-dihydropyrano[2,3-c] imidazo[1,2-a]pyridine,

(7R,8R,9R)-2,3-dimethyl-8-hydroxy-7-ethoxy-9-phenyl-7H-8,9-dihydropyrano[2,3-c]imidazo[1,2-a] pyridine,

(7S,8R,9R)-2,3-dimethyl-8-hydroxy-7-ethoxy-9-phenyl-7H-8,9-dihydropyrano[2,3-c]imidazo[1,2-a] pyridine,

(7R,8R,9R)-2,3-dimethyl-8-hydroxy-7-(2-methoxypropoxy)-9-phenyl-7H-8,9-dihydropyrano[2,3-c] imidazo[1,2-a]pyridine,

(7S,8R,9R)-2,3-dimethyl-8-hydroxy-7-(2-methoxypropoxy)-9-phenyl-7H-8,9-dihydropyrano[2,3-c] imidazo[1,2-a]pyridine,

(7R,8R,9R)-2,3-dimethyl-8-hydroxy-7-(2-propoxy)-9-phenyl-7H-8,9-dihydropyrano[2,3-c]imidazo[1,2-a] pyridine,

(7S,8R,9R)-2,3-dimethyl-8-hydroxy-7-(2-propoxy)-9-phenyl-7H-8,9-dihydropyrano[2,3-c]imidazo[1,2-a] pyridine,

(7R,8R,9R)-2,3-dimethyl-8-hydroxy-7-butoxy-9-phenyl-7H-8,9-dihydropyrano[2,3-c]imidazo[1,2-a] pyridine,

(7S,8R,9R)-2,3-dimethyl-8-hydroxy-7-butoxy-9-phenyl-7H-8,9-dihydropyrano[2,3-c]imidazo[1,2-a] pyridine,

(7S,8R,9R)-7,8-dihydroxy-6-methoxymethyl-2,3-dimethyl-9-phenyl-7,8,9,10-tetrahydroimidazo[1,2-h] [1,7]naphthyridine,

(7R,8R,9R)-7,8-dihydroxy-6-methoxymethyl-2,3-dimethyl-9-phenyl-7,8,9,10-tetrahydroimidazo[1,2-h] [1,7]naphthyridine,

(7S,8R,9R)-8-hydroxy-7-methoxy-6-methoxymethyl-2,3-dimethyl-9-phenyl-7,8,9,10-tetrahydroimidazo[1,2-h][1,7]naphthyridine,

(7R,8R,9R)-8-hydroxy-7-methoxy-6-methoxymethyl-2,3-dimethyl-9-phenyl-7,8,9,10-tetrahydroimidazo[1,2-h][1,7]naphthyridine,

(7R,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,

(7S,8R,9R)-8-Hydroxy-7-(2-methoxyethoxy)-6-methoxymethyl-2,3-dimethyl-9-phenyl-7,8,9,10-tetrahydroimidazo[1,2-h][1,7]naphthyridine,

(7R,8R,9R)-8-hydroxy-7-ethoxy-6-methoxymethyl-2,3-dimethyl-9-phenyl-7,8,9,10-tetrahydro-imidazo[1,2-h][1,7]naphthyridine,

(7S,8R,9R)-8-hydroxy-7-ethoxy-6-methoxymethyl-2,3-dimethyl-9-phenyl-7,8,9,10-tetrahydroimidazo[1,2-h][1,7]naphthyridine,

7,8-dihydroxy-2,3-dimethyl-9-(3-thienyl)-7,8,9,10-tetrahydroimidazo[1,2-h][1,7]naphthyridine, 7-hydroxy-2,3-dimethyl-9-(3-thienyl)-7,8,9,10-tetrahydroimidazo[1,2-h][1,7]naphthyridine, 9-(3-furyl)-7-hydroxy-2,3-dimethyl-7,8,9,10-tetrahydroimidazo[1,2-h][1,7]naphthyridine,

(7R,8R,9R)-8-hydroxy-7-[2-(2-methoxyethoxy)ethoxy]-2,3-dimethyl-9-phenyl-7,8,9,10-tetrahydroimdiazo[1,2-h][1,7]naphthyridine,

(7S,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,

(7R,8R,9R)-7,8-dihydroxy-2-methyl-9-phenyl-7,8,9,10-tetrahydroimidazo[1,2-h][1,7]naphthyridine,

(7S,8R,9R)-8-hydroxy-2-methyl-7-(2-methoxyethoxy)-9-phenyl-7,8,9,10-tetrahydroimidazo[1,2-h][1,7] naphthyridine,

(7R,8R,9R)-8-hydroxy-2-methyl-7-(2-methoxyethoxy)-9-phenyl-7,8,9,10-tetrahydroimidazo[1,2-h][1,7] naphthyridine,

(7R,8R,9R)-3-bromo-8-hydroxy-7-(2-methoxyethoxy)-2-methyl-9-phenyl-7,8,9,10-tetrahydroimidazo-[1,2-h][1,7]naphthyridine,

(7R,8R,9R)-3-chloro-8-hydroxy-7-(2-methoxyethoxy)-2-methyl-9-phenyl-7,8,9,10-tetrahydroimidazo-[1,2-h][1,7]naphthyridine,

(7R,8R,9R)-3-bromo-7-hydroxy-8-(2-methoxyethoxy)-2-methyl-9-phenyl-7,8,9,10-tetrahydroimidazo-[1,2-h][1,7]naphthyridine,

(7R,8R,9R)-3-chloro-8-hydroxy-7-(2-methoxyethoxy)-2-methyl-9-phenyl-7H-8,9-dihydro-pyrano[2,3-c] imidazo[1,2-a]pyridine,

(7R,8R,9R)-8-hydroxy-7-(2-methoxyethoxy)-2-methyl-9-phenyl-7H-8,9-dihydro-pyrano[2,3-c] imidazo[1,2-a]pyridine,

(7R,8R,9R)-7,8-dihydroxy-2-methyl-9-phenyl-7H-8,9-dihydropyrano[2,3-c] imidazo[1,2-a]pyridine,

(7S,8R,9R)-7,8-dihydroxy-2-methyl-9-phenyl-7,8,9,10-tetrahydroimidazo[1,2-h][1,7]naphthyridine,

(7R,8R,9R)-8-hydroxy-7-methoxy-2-methyl-9-phenyl-7,8,9,10-tetrahydroimidazo[1,2-h][1,7] naphthyridine,

(7S,8R,9R)-8-hydroxy-7-methoxy-2-methyl-9-phenyl-7,8,9,10-tetrahydroimidazo[1,2-h][1,7] naphthyridine,

(7R,8R,9R)-3-hydroxymethyl-8-hydroxy-7-(2-methoxyethoxy)-2-methyl-9-phenyl-7,8,9,10-tetrahydroimidazo[1,2-h][1,7]naphthyridine,

(7R,8R,9R)-3-hydroxymethyl-8-hydroxy-7-(2-hydroxyethoxy)-2-methyl-9-phenyl-7,8,9,10-tetrahydroimidazo[1,2-h][1,7]naphthyridine,

(7R,8R,9R)-2,3-dimethyl-8-hydroxy-7-(2-hydroxyethoxy)-9-phenyl-7,8,9,10-tetrahydroimidazo[1,2-h] [1,7]naphthyridine,

(7R,8R,9R)-3,9-diphenyl-8-hydroxy-7-(2-methoxyethoxy)-2-methyl-7,8,9,10-tetrahydroimidazo[1,2-h] [1,7]naphthyridine,

(7R,8R,9R)-7,8-dihydroxy-2-methoxymethyl-3-methyl-9-phenyl-7,8,9,10-tetrahydroimidazo[1,2-h][1,7] naphthyridine,

(7S,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,

(7R,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,

(7S,8R,9R)-7-ethoxy-8-hydroxy-2-methoxymethyl-3-methyl-9-phenyl-7,8,9,10-tetrahydroimidazo[1,2-h] [1,7]naphthyridine,

(7R,8R,9R)-7-ethoxy-8-hydroxy-2-methoxymethyl-3-methyl-9-phenyl-7,8,9,10-tetrahydroimidazo[1,2-h] [1,7]naphthyridine,

(8S)-2,3-Dimethyl-8-phenyl-7,8-dihydro-6H-9oxa-1,3a-diaza-cyclopenta[a]naphthalene-5-carboxylic acid dimethylamid,

8-[(1S,2S)-2,3-dihydro-2-hydroxy-1-indenyloxy-6-(N,N-dimethylaminocarbonyl)-2,3-dimethyl-imidazo[1,2-a]pyridine,

6-(N,N-Dimethylaminocarbonyl)-4-(2,6-dimethyl-benzylamino)-1,2-dimethyl-1H-benzimidazole, and the pharmacologically suitable salts of these compounds.

An example of a preferred proton pump antagonist which may be mentioned is the compound (7R,8R,9R)-2,3-dimethyl-8-hydroxy-7-(2-methoxyethoxy)-9-phenyl-7,8,9,10-tetrahydroimidazo[1,2-h][1,7]naphthyridine (INN: soraprazan).

The proton pump antagonists may in this connection be present as such or in the form of their salts and/or solvates (e.g. hydrates) etc. Most reversible proton pump inhibitors are basic compounds. Particularly suitable salts are all acid addition salts. Particular mention may be made of the pharmacologically acceptable salts of the inorganic and organic acids normally used in pharmaceutical technology. Suitable as such are water-soluble and water-insoluble acid addition salts with acids such as, for example, hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid or 3-hydroxy-2-naphthoic acid, the acids being employed in the preparation of the salts, depending on whether the acid is mono- or polybasic and on which salt is desired—in the equimolar ratio of amounts or one differing therefrom.

Orodispersible dosage form in connection with the invention is to be understood as dosage form, which when placed in the oral cavity disperses rapidly before being swallowed. After disintegration in the oral cavity the tablet constituents are swallowed and the drug substance is absorbed in the GI tract. Dependent on the properties of the drug substance itself and on the formulation, partial or complete absorption via the oral mucosa might occur. The dosage form according to the invention therefore comprises an effective amount of a proton pump antagonist (APA) together with excipients which, on oral intake of the dosage form, bring about rapid disintegration of the dosage form in the oral cavity, and, where appropriate, further excipients. Preferably the dosage form shows an immediate release of the active ingredient.

The dosage forms according to the invention preferably do not show, in contrast to the dosage forms described in EP-0841904-B1, extended release but show immediate release of the active ingredient. Preference is therefore given according to the invention to orally disintegrating dosage form with immediate release of the active ingredient (immediate release solid oral dosage form). The dosage form preferably has a maximum disintegration time in water (at 37° C.) of 3 minutes, 2 minutes or 1 minute. (The disintegration time of the tablet can be determined according to standard procedures disclosed in pharmacopoeia monographs, preferably according to the European Pharmacopoeia 4^{th} edition). The dosage form preferably has a release of active ingredient of greater than or equal to 60% after 15 minutes in 0.1 N hydrochloric acid, particularly preferable greater than or equal to 75% after 15 minutes in 0.1 N hydrochloric acid, more particularly preferable greater than or equal to 80% after 15 minutes in 0.1 N hydrochloric acid and even more particularly preferable greater than or equal to 85% after 15 minutes in 0.1 N hydrochloric acid. In a preferred embodiment the dosage form has a release of active ingredient of greater than or equal to 90% after 15 minutes and preferably a release of active ingredient of greater than or equal to 95% after 30 minutes (label claim).

In one embodiment of the invention, the orodispersible dosage form according to the invention is a dosage form displaying the properties according to the pharmacopoeia monographs in the European Pharmacopoeia 4^{th} edition “Orodispersible tablets”.

In a preferred embodiment the dosage form according to the invention is a rapidly disintegrating dosage form which shows a disintegration time determined in water at 37° C. of not more than 3, 2 or 1 min and a dissolution (release of active ingredient) greater than or equal to 85% after 15 minutes in 0.1 N hydrochloric acid.

The dosage forms according to the invention are distinguished by oral disintegration, rapid release of active ingredient and an optimal action profile (e.g. a rapid onset of action) in the therapy of diseases derived from increased gastric acid secretion. There is furthermore observed to be an improved stability of the proton pump antagonists in dosage forms according to the invention containing a basic excipient. Various methods and technologies are available in the art for providing orodispersible dosage forms. In the art other terms such as fast melt, rapidly disintegrating or orally disintegrating are used synonymously with the term orodispersible. The term orodispersible as used in connection with the present inventions is to be understood to encompass those terms, respectively be interchangeable with those terms. Orodispersible dosage forms can be prepared by several manufacturing methods. Orodispersible dosage forms are for example known from U.S. Pat. No. 5,866,163, U.S. Pat. No. 5,869,098, U.S. Pat. No. 5,178,878, WO 0009090, WO 0009095, WO 9846215, U.S. Pat. No. 5,607,697, U.S. Pat. No. 5,178,878, WO 9944580, WO 0285336, WO 0051568, WO 0027357, FR 2766089, WO 9301805, U.S. Pat. No. 5,762,961, U.S. Pat. No. 4,946,684, WO 0245571, U.S. Pat. No. 5,738,875, U.S. Pat. No. 5,298,261, U.S. Pat. No. 5,466,464, WO 0292057, WO 0202083, WO 9947126, WO 9947124, JP 11033084, WO 9520380, U.S. Pat. No. 5,501,861, WO 0006126, JP 2002145804, WO 0264119, JP 09048726, JP 02255797, WO 0047233, JP 2000119174, JP 02037727, WO 0164190, JP 2001253818, WO 9955311, JP 11012162, WO 9904758, WO 9747287, JP 09071523, JP 11199517, JP 2002138055, JP 2002128661, US 2002122822, WO 0255061, WO 0247607, WO 0189485, WO 0176565, JP 2001163770, WO 0112161, WO 0110418, U.S. Pat. No. 6,066,337, U.S. Pat. No. 6,010,719, WO 0057857 and WO 0119336.

For example orodispersible tablets can be prepared by compression of powders or granules. Normally fast dissolving or fast disintegrating excipients are employed resulting in a pleasant mouth feel and taste. Special formulation technologies based on direct compression have been developed and commercialized by Cima Labs, USA, as described for example in patents U.S. Pat. No. 5,178,878, U.S. Pat. No. 6,024,981, U.S. 6,221,392 and in the literature (Modified-release drug delivery technology, edited by M. Rathbone, J. Hadgraft, M. Roberts, 2003, Marcel Dekker). For the OraSolv™ technology the fast disintegration is achieved by compressing water-soluble excipients using a lower range of compression forces than are normally used in tableting. The low compression force leads to high tablet porosity which accelerates the rate of disintegration of the tablet and dissolution of the water-soluble excipients. Disintegrating agents further facilitate the process, an effervescent couple being used as a water-soluble disintegrating agent. These soft and friable tablets needs to be packaged in special packaging systems.

In contrast the DuraSolv™ technology results in robust yet quick-dissolving tablets. DuraSolv™ utlilizes nondirectly compressible fillers (e.g. powdered mannitol) in fine particle form. These fillers have a high surface area, which increases their dissolution rate. Wicking agents (e.g. crosslinked PVP) assist the entry of water into the body of the tablet. Taste-masked drug particles can be incorporated in the tablets.

Another method to produce orodispersible dosage forms is by lyophilization (eg Zydis technology developed by R P Scherer) as described e.g. in U.S. Pat. No. 5,631,023, U.S. Pat. No. 5,738,875 and other patents to R P Scherer and the literature (Modified-release drug delivery technology, edited by M. Rathbone, J. Hadgraft, M. Roberts, 2003, Marcel Dekker). This process requires the active ingredient to be dissolved or suspended in an aqueous solution of water-soluble structure formers. The resultant mixture is then poured into the preformed blister pockets of a laminate film and freeze-dried. The two most commonly used structural excipients are gelatine and mannitol although other suitable structure formers can be used (e.g. starches, gums, etc.) depending on the properties of the active ingredient.

Dosage form in connection with the invention refers to any suitable orodispersible dosage form preferably to oral solid dosage forms such as tablets, dosage forms based on lyophilised (freeze-dried) preparations and preparations in the form of a thin film.

Basic excipients which are suitable according to the invention and which can be employed in the dosage forms according to the invention to stabilize the proton pump antagonists are substances which have a basic reaction and are pharmacologically acceptable and able to stabilize the proton pump antagonists in the dosage form. These are, in particular, compounds selected from the group of pharmacologically acceptable alkali metal, alkaline earth metal or earth metal salts of weak acids, pharmacologically suitable hydroxides and oxides of alkaline earth and earth metals or else pharmacologically acceptable basic buffer systems. Examples which may be mentioned are sodium carbonate, calcium carbonate, magnesium carbonates, magnesium oxide, magnesium hydroxide, magnesium metasilicate aluminate, magnesium silicates, magnesium aluminate, hydrotalcite (synthetic), aluminium magnesium hydroxide, and calcium hydroxide, basic salts of amino acids, sodium hydroxide, trihydroxymethylaminomethane, trisodium citrate, disodium hydrogen phosphate and trisodium phosphate or mixtures thereof.

Preference is given according to the invention to sodium carbonate, disodium hydrogen phosphate, trisodium phosphate and buffer systems composed of disodium hydrogen phosphate with sodium hydroxide.

If the orodispersible tablet is prepared by compression of powders or granules, the basic excipient is preferably thoroughly mixed in finely divided form with the active ingredient and, where appropriate, other excipients or carriers so that there is intensive (direct) contact between basic excipient and the active ingredient. A further possibility is also to employ excipient granules impregnated with a basic buffer system.

The basic excipient is preferably added in an amount such that when 100 mg of mixtures of the active ingredient with the desired excipients are dissolved in 50 ml of purified water the basicity reaches not less than pH 7, preferably a basicity of pH 8 to pH 11.5, particularly preferably of pH 8 to pH 11.0 and very particularly preferably of pH 8.5 to 10.5. Depending on the nature of the basic excipient, the content can therefore be for example from 0.1 to 30% by weight (in percent by weight based on the finished dosage form). In a preferred embodiment the content of the basic excipient is below 20% by weight, particularly preferable below 15% by weight and in particular below 10% by weight (in percent by weight based on the finished dosage form).

Depending on the dosage form in addition to the basic excipients further suitable excipients can be present, for example, excipients which bring about rapid disintegration of the dosage form in the oral cavity. Further examples of excipients which may be mentioned are fillers, carriers, disintegrants, binders, effervescence systems, lubricants, colouring agents, sweeteners, flavourings, pH-modifier and surface-active substances.

In one embodiment the dosage form according to the invention is a tablet. Further subject of the invention is therefore an oral dosage form for proton pump antagonists (APA) comprising an effective amount of a proton pump antagonist together with excipients, which dosage form is an orodispersible tablet. Further subject of the invention is an oral dosage form for proton pump antagonists (APA) comprising an effective amount of a proton pump antagonist together with excipients, which dosage form is an orodispersible tablet and wherein the proton pump antagonist is stabilized in the dosage form by one or more basic excipients.

In connection with the tablet according to the invention further excipients which are used are, for example, excipients which bring about rapid disintegration of the dosage form in the oral cavity. These preferably comprise one or more substances selected from the group of fillers or carriers. It is furthermore possible for one or more excipients from the group of disintegrants, binders, effervescence systems, lubricants, colouring agents, sweeteners, flavourings, pH-modifier and surface-active substances to be present.

Fillers or carriers suitable in connection with the tablet according to the invention are, in particular, fillers such as, calcium silicate (Rxipients®), sugar alcohols such as mannitol (e.g. Perlitol® or Parteck® M, Merck, Germany), sorbitol (e.g. Karion®), xylitol, erythritol (e.g. Erythritol DC, Cerestar, Belgium), or maltitol, starches such as corn starch, potato starch and wheat starch, microcrystalline cellulose, saccharides such as glucose, lactose, levulose, sucrose and dextrose, co-processed fillers such as Pharmaburst®, SPI Pharma, USA, Starlac™, Meggle, Germany.

The content (in percent by weight based on the finished dosage form) of filler in the tablet according to the invention is advantageously from 1 to 99% by weight. The content of filler is preferably from 30 to 95% by weight, and the content is particularly preferably from 60 to 90% by weight.

If appropriate disintegrants can be added. Disintegrants suitable according to the invention are, in particular, insoluble polyvinylpyrrolidone (insoluble PVP, crosspovidone), sodium carboxymethyl starch, croscarmellose sodium, alginic acid, and starches able to fulfil the function of a disintegrant (e.g. Starch 1500).

The content (in percent by weight based on a tablet according to the invention) of disintegrant in the orodispersible tablet according to the invention can usually be from 0.5 to 30% by weight. The content of disintegrant is preferably from 1 to 15% by weight. The content of disintegrant is particularly preferably from 1 to 5% by weight.

Suitable lubricants which may be mentioned are sodium stearyl fumarate, magnesium stearate, calcium stearate, stearic acid, talc and colloidal silica (Aerosil).

The content (in percent by weight based on the finished dosage form) of lubricant in the orodispersible tablet according to the invention is usually from 0.1 to 5% by weight. The content of lubricant is preferably from 0.2 to 3% by weight. The content of lubricant is particularly preferably from 0.5 to 2% by weight.

Binders suitable according to the invention are polyvinylpyrrolidone (PVP, Polyvidon® K25, Polyvidon® K90) or mixtures of PVP with polyvinyl acetate (e.g. Kollidon® 64), gelatin, corn starch paste, preswollen starches (Starch® 1500, Uni-Pure® WG220), hydroxypropylmethylcellulose (HPMC) or hydroxypropylcellulose (L-HPC).

The content (in percent by weight based on the tablet according to the invention) of binder can be up to 10% by weight, and it can preferably be up to 5% by weight.

Suitable surface-active substances which may be mentioned are sodium lauryl sulfate or Tween® 20, Tween® 60 or Tween® 80.

The tablet according to the invention particularly preferably contains a mixture of at least one basic excipient, filler or carrier, one lubricant, sweeteners and flavouring agents.

It is also possible if desired for one or more flavours and sweeteners to be present in the dosage form according to the invention. It is possible thereby for example to achieve an improvement in taste. These substances are added in the usual amounts.

In the case of proton pump antagnoists which are photosensitive, a colouring agent may be included in the process to produce the tablet cores, and the solid dosage form is coloured. Colouring agents which may be mentioned are, for example, iron oxides, Indigocarmin E132 or titanium dioxide. These can be processed directly in the mixture with the active ingredient to give coloured dosage forms.

The dosage form according to the invention which is a tablet is produced by processes known to the skilled person, in particular by mixing the proton pump antagonists with the excipients. It is preferred in this connection for the active ingredient to be mixed thoroughly with the basic excipients. In the case of direct compressed tablets, the orodispersible tablet is preferably produced by dry mixing of the excipients with the active ingredient. If desired, the active ingredient can be premixed with part of the filler or carrier. Conventional mixers such as compulsory mixers or free-fall mixers can be employed for the mixing operation. The preparations obtained in this way can then be compressed on a suitable tablet press applying compaction forces. If desired, precompaction may also take place. In the case of coloured dosage forms the colouring agent is preferably admixed dry.

In a preferred embodiment according to the invention the dosage form is a tablet, comprising (7R,8R,9R)-2,3-dimethyl-8-hydroxy-7-(2-methoxyethoxy)-9-phenyl-7,8,9,10-tetrahydroimidazo[1,2-h][1,7]naphthyridine (INN soraprazan) or a pharmacologically acceptable salt and/or hydrate thereof as proton pump antagonist and sodium carbonate as basic excipient. Preferably the dosage form shows a disintegration time determined in water at 37° C. of not more than 3 minutes, 2 minutes or 1 minute and dissolution (release of active ingredient) greater than or equal to 85% after 15 minutes in 0.1 N hydrochloric acid.

In another aspect according to the invention the orodispersible dosage form is a dosage form based on lyophilised (freeze-dried) preparations. In connection with the lyophilised preparation according to the invention further excipients which are used are, for example, basic excipients as stated above and excipients which bring about rapid disintegration of the dosage form in the oral cavity. Examples which may be mentioned are in particular water-soluble structure formers. Commonly used structural excipients are gelatine and mannitol although other suitable structure formers can be used (e.g. starches, gums, etc.) depending on the properties of the active ingredient. Flavours and sweeteners can be added in order to improve the taste.

The dosage form according to the invention is produced by processes known to the skilled person, in particular by mixing the proton pump antagonists with the excipients. It is preferred in this connection for the active ingredient to be mixed thoroughly with the basic excipients preferably by suspending or dissolving the basic excipients together with the proton pump antagonist in the aqueous solution of excipients prior to the freeze-drying.

In the case of dosage forms based on lyophilized preparations the dosage form is preferably produced by dissolving or suspending the active in an aqueous solution of excipients, filling the solution or suspension in blister pockets, freezing the solution or suspension, removing the ice by sublimation and sealing the blister pockets.

Since palatability is an important issue for orodispersible dosage form taste-masking may be required. In the case of proton pump antagonists with unpleasant taste a taste masking of the active ingredient is realized by a technology appropriate to assure a palatable formulation, e.g. fluid bed coating, micro-encapsulation, embedding or spray congealing. In this case coated particles comprising the active ingredients such as pellets, granules and crystals may be used for manufacturing the dosage forms according to the invention. Coating of particles may be achieved in the case of pellets for example by spraying a basified active ingredient preparation onto starter pellets or pellets prepared by the extruder/spheronizer process.

The following formulation examples illustrate the invention in detail without restricting it.

### EXAMPLE

### Example 1

Direct Compressed Tablet:

| | | | |
|---|---|---|---|
| a) | Soraprazan | 20.00 | mg |
| b) | Sodium carbonate | 5.10 | mg |
| c) | Mannitol | 133.84 | mg |
| d) | Rxcipients | 23.90 | mg |
| e) | Microcrystalline cellulose | 35.85 | mg |
| f) | Crospovidone | 2.39 | mg |
| g) | Sodium starch glycolate | 2.39 | mg |
| h) | Colloidal Silicon dioxide | 2.39 | mg |
| i) | Saccharose | 4.78 | mg |
| j) | Flavour | 7.16 | mg |
| k) | Magnesium stearate | 1.20 | mg |
| | | 239.00 | mg |

a) is premixed with e) in a compulsory mixer. This mixture is admixed with b), c) d), f), g), h), i), and j) in the compulsory mixer. Subsequently k) is admixed in a free-fall mixer. The tabletting mixture is compressed to tablets in a suitable tablet press.

### Example 2

Direct Compressed Tablets

| | | | |
|---|---|---|---|
| a) | Soraprazan | 20.00 | mg |
| b) | Sodium carbonate | 5.10 | mg |
| c) | Mannitol | 154.99 | mg |
| d) | Rxcipients | 60.99 | mg |
| f) | Crospovidone | 6.78 | mg |
| g) | Aspartam | 0.75 | mg |
| h) | Flavour | 0.50 | mg |
| i) | Sodium stearyl fumarate | 1.89 | mg |
| | | 251.00 | mg |

a)-h) are mixed in a compulsory mixer. Subsequently i) is admixed in a free-fall mixer. The tabletting mixture is compressed to tablets in a suitable tablet press.

### Example 3

Direct Compressed Tablets

| | | | |
|---|---|---|---|
| a) | Soraprazan | 20.00 | mg |
| b) | Sodium carbonate | 5.10 | mg |
| c) | Microcrystalline cellulose | 155.62 | mg |
| d) | Rxcipients | 67.77 | mg |
| e) | Aspartam | 0.75 | mg |
| f) | Flavour | 0.50 | mg |
| g) | Magnesium stearate | 1.26 | mg |
| | | 251.00 | mg |

a) is premixed with a part of c) are mixed in a compulsory mixer. This mixture is admixed with b), the rest of c), d), e) and f) in the compulsory mixer. Subsequently g) is admixed in a free-fall mixer. The tabletting mixture is compressed to tablets in a suitable tablet press.

### Example 4

Direct Compressed Tablets

| | | | |
|---|---|---|---|
| a) | Soraprazan | 20.000 | mg |
| b) | Sodium carbonate | 5.100 | mg |
| c) | Pharmaburst B2 ™ | 219.900 | mg |
| d) | Aspartam | 0.375 | mg |
| e) | Acesulfam Potassium | 0.375 | mg |
| f) | Flavour | 0.500 | mg |
| g) | Magnesium stearate | 3.750 | mg |
| | | 250.000 | mg |

a)-f) are mixed in a compulsory mixer. Subsequently g) is admixed in a free-fall mixer. The tabletting mixture is compressed to tablets in a suitable tablet press.

### Example 5

Direct Compressed Tablets

| | | | |
|---|---|---|---|
| a) | Soraprazan | 20.000 | mg |
| b) | Sodium carbonate | 5.100 | mg |
| c) | Starlac ™ | 222.400 | mg |
| d) | Aspartam | 0.375 | mg |
| e) | Acesulfam Potassium | 0.375 | mg |
| f) | Flavour | 0.500 | mg |
| g) | Magnesium stearate | 1.250 | mg |
| | | 250.000 | mg |

a)-f) are mixed in a compulsory mixer. Subsequently g) is admixed in a free-fall mixer. The tabletting mixture is compressed to tablets in a suitable tablet press.

### Example 6

Direct Compressed Tablets

| | | | |
|---|---|---|---|
| a) | Soraprazan | 20.000 | mg |
| b) | Sodium carbonate | 5.100 | mg |
| c) | Croscarmellose Na | 10.00 | mg |
| d) | Granulated mannitol | 209.900 | mg |
| | (eg Mannogem granular ™, Partek ™) | | |
| e) | Aspartam | 0.375 | mg |
| f) | Acesulfam Potassium | 0.375 | mg |
| g) | Flavour | 0.500 | mg |
| h) | Magnesium stearate | 3.750 | mg |
| | | 250.000 | mg |

a)-g) are mixed in a compulsory mixer. Subsequently h) is admixed in a free-fall mixer. The tabletting mixture is compressed to tablets in a suitable tablet press.

### Example 7

Direct Compressed Tablets

| | | | |
|---|---|---|---|
| a) | Soraprazan | 20.000 | mg |
| b) | Sodium carbonate | 5.100 | mg |
| c) | Croscarmellose Na | 10.00 | mg |
| d) | Erythritol DC ™ | 209.900 | mg |
| e) | Aspartam | 0.375 | mg |
| f) | Acesulfam Potassium | 0.375 | mg |
| g) | Flavour | 0.500 | mg |
| h) | Magnesium stearate | 3.750 | mg |
| | | 250.000 | mg |

a)-g) are mixed in a compulsory mixer. Subsequently h) is admixed in a free-fall mixer. The tabletting mixture is compressed to tablets in a suitable tablet press.

### Example 8

Direct Compressed Tablet

| | | | |
|---|---|---|---|
| a) | Soraprazan | 20.000 | mg |
| b) | Sodium carbonate | 5.100 | mg |
| c) | Crosslinked PVP (as wicking agent) | 15.000 | mg |
| d) | Powdered mannitol | 152.900 | mg |
| | (as non-direct compression filler) | | |
| e) | Aspartam | 0.375 | mg |
| f) | Acesulfam Potassium | 0.375 | mg |
| g) | Flavour | 0.500 | mg |
| h) | Colloidal silicone dioxide | 0.750 | mg |
| i) | Magnesium stearate | 5.000 | mg |
| | | 200.000 | mg |

a)-h) are mixed in a compulsory mixer. Subsequently i) is admixed in a free-fall mixer. The tabletting mixture is compressed to tablets in a suitable tablet press.

### Example 9

Tablet Formulation Employing Taste-Masked Pellets

1. 1.2 g of HPMC 5cp and 0.15 g of sodium laurylsulphate will be dissolved in 46.35 g of purified water. 0.800 g of soraprazan and 0.200 g of sodium carbonate will be suspended under stirring. The suspension will be sprayed onto 28.5 g of pellets made out of microcrystalline cellulose, eg Cellets™, Cellspheres™ employing a Wurster tube. The particle size of the pellets will be approx. 200 μm.

2. In a second process step a coating suspension will be sprayed on the drug containing pellets prepared under 1. in order to mask the taste of soraprazan. 1.51 g of sodium dodecylsulfate will be dissolved in 125.87 g of water by stirring. After 5 min stirring 15.08 g of Eudragit EPO will be suspended. After another 10 min 2.26 g of stearic acid will be added and the suspension will be stirred for at least 5 hours. 25 g of the pellets prepared under 1. will be fluidized employing a Wurster-tube. 37.5 g of the granulation suspension will be sprayed upon the pellets.

Resulting pellets can be compressed to tablets by direct compression as outlined in the examples above. In this case there is no need to add sodium carbonate to the powder mixture since this is already included in the pellets.

### Example 10

Orodispersible Dosage Form Prepared by Lyophilization

| | | | |
|---|---|---|---|
| a) | Soraprazan | 0.800 | g |
| b) | Sodium carbonate | 0.204 | g |
| c) | Gelatin | 2.345 | g |
| d) | Mannitol | 1.759 | g |
| e) | Aspartam | 0.300 | g |
| f) | Flavour | 0.150 | g |
| g) | Purified water | 54.500 | g |

Gelatin will be added to water and heated with mixing to approximately 40° C. The gelatin solution will be added to a mixture of mannitol, sodium carbonate and soraprazan and will be stirred until homogenously suspended. The mixture will be cooled under vacuum and aspartame and flavour will be added. The suspension will be dosed into the blister pockets, frozen and freezedried to produce the final dosage form.

Stability Testing

Triturations of soraprazan with different excipients including or excluding a basic excipient were manufactured, stored at 50° C. and analysed for impurities. The following results were obtained:

| | Mixture | | | | | |
|---|---|---|---|---|---|---|
| | | | | Soraprazan, | Soraprazan, | |
| | | | | Corn | Corn | Soraprazan, |
| | | | | Starch, | Starch, | Mannit |
| | | | | Mannit, | Magnesium | Magnesium |
| | | | | Magnesium | Stearate, | Stearate, |
| | | Soraprazan, | | Stearate, | Sodium | Sodium |
| | Soraprazan, | Magnesium | Soraprazan, | Disodium | Hydrogen- | Hydrogen- |
| | Mannit | Stearate | Corn Starch | Carbonate | carbonate | carbonate |
| Impurities | 5.29 | 5.01 | 6.67 | 3.76 | 3.68 | 3.74 |
| total | | | | | | |
| (AU %) | | | | | | |

### INDUSTRIAL APPLICABILITY

Proton pump antagonists and their salts have valuable pharmacological properties which make them industrially utilizable. They show in particular a pronounced inhibition of gastric acid secretion and an excellent gastrointestinal-protective effect in warm-blooded species, especially humans. The compounds according to the invention are distinguished in this connection by a high selectivity of effect, an advantageous duration of action, a particularly good enteral activity, the absence of substantial side effects and a high therapeutic index.

“Gastrointestinal protection” means in this connection the prevention and treatment of gastrointestinal disorders, especially gastrointestinal inflammatory disorders and lesions (such as, for example, gastric ulcer, duodenal ulcer, gastritis, hyperacidic or drug-related dyspepsia, heartburn and acid eructation, severe reflux oesophagitis, prophylaxis of recurrent reflux oesophagitis and of duodenal ulcer, reflux oesophagitis, Zollinger-Ellison syndrome, elimination of the pathogen Helicobacter pylori in combination with amoxicillin and clarithromycin or in combination with clarithromycin and metronidazole or with amoxicillin and metronidazole, long-term treatment for prophylaxis of recurrent severe forms of reflux oesophagitis. Prophylaxis and therapy of ulcers and gastroduodenal erosions induced by non-steroidal antiinflammatory drugs), which may be caused for example by microorganisms (e.g. Helicobacter pylori), bacteriotoxins, medicines (e.g. certain antiinflammatory and antirheumatic drugs), chemicals (e.g. ethanol), gastric acid or stress situations.

Owing to these properties, the dosage forms according to the invention containing a proton pump antagonist and/or a pharmacologically acceptable salt thereof are outstandingly suitable for use in human and veterinary medicine, being used in particular for the treatment and/or prophylaxis of disorders of the stomach and/or intestine.

The invention therefore further relates to the dosage forms according to the invention for use in the treatment and/or prophylaxis of the aforementioned disorders.

The invention also includes the use of the dosage forms according to the invention for the treatment and/or prophylaxis of the aforementioned disorders. The dosage forms according to the invention may in this case be employed as such (e.g. direct oral intake by the patient) or be dissolved or dispersed in water before use. The solutions or suspensions obtained after dispersion in a suitable dispersant or solvent can then be taken by the patient. This may, for example, be advantageous for patients who have problems with taking a solid dosage form. A further possibility is to administer such solutions or suspensions also by means of tubes (e.g. nose tubes, stomach tube). This is advantageous in particular on administration of the dosage forms according to the invention in patients receiving intensive care, patients with swallowing difficulties, bedridden patients and children.

The dosage forms according to the invention can be combined with other medicaments, either in different combinations or in a fixed combination. Combinations worth mentioning in connection with the dosage forms according to the invention containing proton pump antagonists as active ingredients are those with antimicrobial active ingredients and those with NSAIDs (non steroidal anti inflammatory drugs). Particular mention should be made of the combination with antimicrobial agents like those employed to control the microbe Helicobacter pylori (H. pylori). Further examples which may be mentioned of combinations are: tranquilizers (for example from the group of benzodiazepines, e.g. diazepam), spasmolytics (e.g. bietamiverine or camylofin), anticholinergics (e.g. oxyphencyclimine or phencarbamide), local anesthetics (e.g. tetracaine or procaine), where appropriate also enzymes, vitamins or amino acids. Combinations of the compounds according to the invention with drugs which inhibit acid secretion should be particularly emphasized in this connection, such as, for example, antacids, H2 blockers (e.g. cimetidine, ranitidine), H+/K+-ATPase inhibitors (e.g. omeprazole, pantoprazole), or else with so-called peripheral anticholinergics (e.g. pirenzepine, telenzepine) and with gastrin antagonists with the aim of enhancing the main effect in an additive or superadditive sense and/or of eliminating or reducing the side effects.

Examples of suitable antimicrobial active ingredients (active against Helicobacter pylori) are described in EP-A-0 282 131. Examples which may be mentioned of antimicrobial agents suitable for controlling the microbe Helicobacter pylori are for example bismuth salts [e.g. bismuth subcitrate, bismuth subsalicylate, ammonium bismuth(III) potassium citrate dihydroxide, bismuth nitrate oxide, dibismuth tris(tetraoxodialuminate)], especially β-lactam antibiotics, for example penicillins (such as benzylpenicillin, phenoxymethylpenicillin, propicillin, azidocillin, dicloxacillin, flucloxacillin, oxacillin, amoxicillin, bacampicillin, ampicillin, mezlocillin, piperacillin or azlocillin), cephalosporins (such as cefadroxil, cefaclor, cefalexin, cefixime, cefuroxime, cefetamet, cefadroxil, ceftibuten, cefpodoxime, cefotetan, cefazolin, cefoperazone, ceftizoxime, cefotaxime, ceftazidime, cefamandole, cefepime, cefoxitin, cefodizime, cefsulodin, ceftriaxone, cefotiam or cefmenoxime) or other β-lactam antibiotics (e.g. aztreonam, loracarbef or meropenem); enzyme inhibitors, for example sulbactam; tetracyclines, for example tetracycline, oxytetracycline, minocycline or doxycycline; aminoglycosides, for example tobramycin, gentamicin, neomycin, streptomycin, amikacin, netilmicin, paromomycin or spectinomycin; amphenicols, for example chloramphenicol or thiamphenicol; lincomycins and macrolide antibiotics, for example clindamycin, lincomycin, erythromycin, clarithromycin, spiramycin, roxithromycin or azithromycin; polypeptide antibiotics, for example colistin, polymixin B, teicoplanin or vancomycin; gyrase inhibitors, for example norfloxacin, cinoxacin, ciprofloxacin, pipemidic acid, enoxacin, nalidixic acid, pefloxacin, fleroxacin or ofloxacin; nitroimidazoles, for example metronidazole; or other antibiotics, for example fosfomycin or fusidic acid. Administration of a reversible proton pump inhibitor together with the combination of a plurality of antimicrobial active ingredients is particularly worthy of mention in this connection, for example with the combination of a bismuth salt and/or tetracycline with metronidazole or the combination of amoxicillin or clarithromycin with metronidazole and amoxicillin with clarithromycin.

The dosage of the active ingredients in the dosage form according to the invention depends greatly on the nature of the proton pump antagonists used. A typical dosage for a proton pump antagonist as disclosed for example in WO-A-9418199 can be regarded as a daily dose of about 0.01 to about 20, preferably about 0.05 to 5, in particular 0.1 to 1.5, mg/kg of body weight, where appropriate in the form of a plurality of single doses. In the case of the compound soraprazan, examples of dosage forms according to the invention contain the proton pump antagonist in a dose of 2, 2.5, 5, 10, 15, 20 or 40 mg.

Antimicrobial active ingredients which may be emphasized are erythromycin, azithromycin, clarithromycin, clindamycin, rifampicin, ampicillin, mezlocillin, amoxicillin, tetracycline, minocycline, doxycycline, imipenem, meropenem, cefalexin, cefuroxime axetil, cefpodoxime proxetil, cefaclor, cefadroxil, ciprofloxacin, norfloxacin, ofloxacin and pefloxacin.

Antimicrobial active ingredients which may be particularly emphasized are clarithromycin and amoxicillin.

Combined administration for the purposes of the present invention mean fixed and, in particular, free combination, i.e. either the proton pump antagonist and the antimicrobial active ingredient are present here in one dosage unit, or the proton pump antagonist and antimicrobial active ingredient, which are present in separate dosage units, are administered in direct succession or at a relatively large interval in time, a relatively large interval in time meaning a time span not exceeding 24 hours. For use as separate dosage units, these are preferably provided in a common package. For example, the two dosage units are packaged together in blisters which are designed in respect of the relative disposition of the two dosage units, the labelling and/or colouring in a manner known per se so that the time that the individual components (dosage regimen) of the two dosage units should be taken are evident to the patient.

1. An oral dosage form for proton pump antagonists (APA) comprising an effective amount of a proton pump antagonist together with excipients, which dosage form is an orodispersible dosage form.

2. The oral dosage form for proton pump antagonists (APA) according to claim 1, comprising an effective amount of a proton pump antagonist together with excipients, where the proton pump antagonist is stabilized in the dosage form by one or more basic excipients.

3. The oral dosage form according to claim 1, wherein excipients which, on oral intake of the dosage form, bring about rapid disintegration of the dosage form in the oral cavity.

4. The oral dosage form according to claim 1, wherein the dosage form is a tablet.

5. The oral dosage form according to claim 4, wherein the basic excipient is present in finely divided form and thoroughly mixed with the proton pump antagonist.

6. The oral dosage form according to claim 1, which is a lyophilized preparation.

7. The oral dosage form according to claim 1, which is a rapidly disintegrating dosage form with immediate release of the active ingredient (immediate release solid oral dosage form).

8. The oral dosage form according to claim 7, which shows a release of active ingredient of greater than or equal to 85% after 15 minutes in 0.1 N hydrochloric acid.

9. The oral dosage form according to claim 4, wherein in that one or more substances selected from the group consisting of fillers and carriers are present as excipients which bring about rapid disintegration of the tablet.

10. The oral dosage form according to claim 9, wherein one or more further excipients selected from the group consisting of disintegrants, lubricants, colouring agents, flavourings and surface-active substances are present.

11. The oral dosage form according to claim 6, wherein one or more water-soluble structure former is present as an excipient.

12. The oral dosage form according to claim 11, wherein at least one excipient selected from the group consisting of gelatine, mannitol and xanthan gum is present.

13. The oral dosage form according to claim 2, characterized in that the basic excipient is selected from the group consisting of sodium carbonate, calcium carbonate, magnesium carbonates, magnesium oxide, magnesium hydroxide, magnesium metasilicate aluminate, magnesium silicates, magnesium aluminate, hydrotalcite (synthetic), aluminium magnesium hydroxide, calcium hydroxide, basic salts of amino acids, sodium hydroxide, trihydroxymethylaminomethane, trisodium citrate, disodium hydrogen phosphate, trisodium phosphate and mixtures thereof.

14. The oral dosage form according to claim 13, comprising sodium carbonate.

15. The oral dosage form according to claim 13, comprising disodium hydrogen phosphate, trisodium phosphate or buffer systems composed of disodium hydrogen phosphate and sodium hydroxide.

16. The oral dosage form according to claim 1, characterized in that a compound selected from the group consisting of AU-461, soraprazan (BYK61359), DBM-819, KR-60436, T-330, YH-1885, YJA-20379-8 and 2,3-dimethyl-8-(2-ethyl-6-methylbenzylamino)imidazo[1,2-a]pyridine-6-carboxamide is present as reversible proton pump inhibitor.

17. The oral dosage form according to claim 16, characterized in that (7R,8R,9R)-2,3-dimethyl-8-hydroxy-7-(2-methoxyethoxy)-9-phenyl-7,8,9,10-tetrahydroimidazo[1,2-h][1,7]naphthyridine (INN soraprazan) or a pharmacologically acceptable salt and/or hydrate thereof is present as proton pump antagonist.

18. The oral dosage form according to claim 4, comprising (7R,8R,9R)-2,3-dimethyl-8-hydroxy-7-(2-methoxyethoxy) -9-phenyl-7,8,9,10-tetrahydroimidazo[1,2-h][1,7]naphthyridine (INN soraprazan) or a pharmacologically acceptable salt and/or hydrate thereof as proton pump antagonist and sodium carbonate as basic exipient.

19. A method for preparing a dosage form according to claim 4 comprising the step of thoroughly mixing the active ingredient with a basic excipient.

20. A method for preparing a dosage form according to claim 4 comprising the steps of mixing the proton pump antagonist with excipients and compressing the mixture on a suitable tablet press and applying compaction forces.

21. A method for preparing a dosage form according to claim 6, comprising the steps of dissolving or suspending the proton pump antagonist in an aqueous solution of excipients, filling the solution or suspension in blister pockets, freezing the solution or suspension, removing the ice by sublimation and sealing the blister pockets.

22. A method for preparing a dosage form according to claim 1 comprising the step of providing the proton pump antagonist in the form of coated particles selected from the group consisting of pellets, granules and crystals.
